(19) United States
(12) Patent Application Publication
(10) Pub. No.: US 2019/0159727 A1
(43) Pub. Date: May 30, 2019

(54) SENSOR-ENABLED FOOTWEAR; SENSORS, INTERFACES AND SENSOR SYSTEMS FOR DATA COLLECTION

(71) Applicant: Sensoria Inc., Redmond, WA (US)

(72) Inventors: Maurizio Macagno, Redmond, WA (US); Davide Vigano, Redmond, WA (US); Christopher Small, Seattle, WA (US); Maria Pia Carmagnani, Redmond, WA (US); Matthew Kueper, Indianola, WA (US); Mario Esposito, Redmond, WA (US)

(21) Appl. No.: 16/067,999

(22) PCT Filed: Dec. 23, 2016
(86) PCT No.: PCT/US2016/068499

§ 371 Date: Jul. 3, 2018

Related U.S. Application Data
(63) Continuation-in-part of application No. 15/540,404, filed on Jun. 28, 2017, now abandoned.
(60) Provisional application No. 62/274,707, filed on Jan. 4, 2016.
(60) Provisional application No. 62/163,861, filed on May 19, 2015.
(60) Provisional application No. 62/099,099, filed on Dec. 31, 2014.

(51) Int. Cl. A61B 5/00; A63B 24/00; A61B 5/053; A61B 5/0205; A61B 5/01; A61B 5/0488; A61B 5/145; A61B 5/11; A43B 3/00; A43B 1/00; A41B 11/00

(52) U.S. Cl. CPC A61B 5/6807; A61B 5/02438; A61B 5/0533; A61B 5/443; A61B 5/02055; A61B 5/01; A61B 5/0488; A61B 5/14542; A61B 5/1112; A43B 3/0005; A43B 3/001; A43B 1/0054; A41B 11/00; A63B 2024/0071; A63B 24/0062

(57) ABSTRACT

Sensing devices including sensors such as flexible and stretchable fabric-based pressure sensors, may be associated with or incorporated in garments, such as socks, intended to be worn against a body surface (directly or indirectly). Specific manifestations of a sensing system incorporated in a sock substrate are described in detail. Dedicated electronic devices interface electrically with sensors through intermediate conductive traces, optional conductive bridges, conductive contacts provided in a mounting tab.

### REFERENCE TO RELATED APPLICATIONS

This application claims priority to U.S. Patent Application No. 62/274,707, filed Jan. 4, 2016. The disclosure of this priority application is incorporated herein by reference in its entirety.

### FIELD

The present invention relates generally to sensors, including pressure sensors associated with or incorporated in footwear intended to contact a body surface (directly or indirectly), and to sensor interfaces with electronic components and devices. Several aspects of detailed sensor system components and integration of sensing systems embodied in footwear are disclosed.

### BACKGROUND

Various types of sensing systems have been incorporated in shoes, insoles, socks and other types of garments for monitoring various physiological parameters for various applications, including recreational, fitness, sporting, military, diagnostic and medical applications. The use of sensing systems for fitness applications to monitor and analyze activities such as running, walking, energy expenditure, and the like, is now common. Medical applications for sensing pressure, temperature and the like for purposes of monitoring neuropathic and other degenerative conditions with the goal of alerting an individual and/or medical service providers to sensed parameters that may indicate the worsening of a condition, lack of healing, and the like, have been proposed. Footwear-related sensing systems directed to providing sensory data for patients suffering from neuropathy, for gait analysis, rehabilitation assessment, shoe research, design and fitting, orthotic design and fitting, and the like, have been proposed.

Sensing devices and footwear having sensors incorporated for monitoring pressure and other body parameters have been proposed. Various types of sensing systems for monitoring various physiological parameters have been incorporated in bands, wrist-worn devices, portable electronic devices, medical devices, shoes, insoles, socks and other types of garments for various applications. The use of sensing systems for fitness applications to monitor and analyze activities such as running, walking, energy expenditure, and the like, is now common. Medical applications for sensing pressure, posture, gait, temperature and the like for purposes of monitoring neuropathic and other degenerative conditions with the goal of alerting an individual and/or medical service providers to sensed parameters that may indicate the worsening of a condition, lack of healing, and the like, have been proposed. Footwear-related sensing systems directed to providing sensory data for patients suffering from neuropathy, for gait analysis, rehabilitation assessment, shoe research, design and fitting, orthotic design and fitting, and the like, have been proposed.

In one aspect, the components and assemblies for collection and analysis of data from sites such as feet and other body surfaces described herein are directed to providing intermittent and/or continuous monitoring and reporting of conditions and activity parameters (such as force, pressure, shear, etc.) at body locations and combining that data with additional data for purposes of analyzing and reporting activity parameters and providing feedback to the user or a third party. In yet other aspects, sensors, interfaces, systems and materials described herein for collection and analysis of physiological and biomechanical data from sites such as feet and other body parts may be used for a variety of sports-related, military, fitness, medical, diagnostic and therapeutic purposes.

### SUMMARY

In one aspect, sensor systems of the present invention comprise one or more sensor(s) mounted to or incorporated in or integrated with or associated with (referred to herein, collectively, as “associated with”) a substrate material incorporated in footwear. In one aspect, sensors are capable of sensing a physiological parameter of the underlying skin or tissue; in some aspects, sensors are capable of sensing force and/or pressure and/or shear exerted on or against an underlying skin or tissue. In some aspects, sensors are capable of sensing conductive impulses or properties associated with a body surface or tissue. Each sensor is electrically connected, optionally via one or more flexible leads, to a flexible and conductive trace associated with the substrate, and conductive traces terminate at conductive signal transfer terminals associated with the substrate. Each sensor may additionally be connected to a ground trace terminating at a ground terminal.

Sensor systems and sensing devices described herein preferably comprise at least one flexible sensor (or means for sensing), and one or more of the sensor(s), flexible leads, and conductive traces may be stretchable and/or elastic as well as being flexible. In some embodiments, the sensor(s), flexible leads and conductive traces may all comprise pliable, electrically resistive and/or conductive fabric materials or fibers. Footwear and other types of substrates incorporating such sensor systems and sensing devices may be comfortably worn by users, and/or contact a body surface of users, under many conditions, providing real time monitoring of conditions at or near body surfaces.

The signal transfer terminal(s) on the substrate may be matingly received in signal receipt terminals associated with a Dedicated Electronic Device (DED) that is mountable to the substrate or electrically connects to the signal transfer terminal(s) and serves as a (temporary or permanent) data collection device. The DED may also (optionally) house batteries or other energy storage (and/or energy generating devices) and serve as a sensor charging device. The DED may additionally communicate with one or more external electronic device(s), such as a smartphone, personal computing device/display, host computer, base station (or hub) or the like for signal transfer, processing, analysis and display to a user and/or others. In some embodiments, the DED may also communicate with other DEDs located in proximity, via the creation of a dynamic “mesh network”, to facilitate the communication and transport of data to the external electronic devices(s), such as a smartphone, personal computing device/display, host computer, base station (or hub). In some embodiments, the external electronic device, and/or the DED, communicates with an external, hosted computing system (operated, e.g., at a centralized, hosted facility and/or in the “Cloud”) that provides additional data analysis, formulates feedback, notifications, alerts, and the like, that may be displayed to the user, a coach, a caretaker, a clinician, or the like, through one or more computing and/or display devices. In alternative embodiments, the DED may itself perform signal processing and analysis, and may display or otherwise communicate feedback directly to a user without interfacing with an external computing device. In some embodiments, the DED is detachably attachable to signal receipt terminals incorporated in an interface component associated with a substrate.

In some embodiments, one or more resistive sensor(s) detect changes in voltage or resistance across a surface area that is associated with force exerted on the sensor, which is related to pressure (as force per unit surface area) and/or shear. Force, pressure, shear and measurements or values that are derivative thereof may therefore be determined at identifiable spatial locations where sensors are positioned. E-textile sensors capable of providing proportional pressure signals (e.g., proportional pressure sensed over a surface area), and/or providing pressure signals that correlate with spatial locations on a surface area of the e-textile sensors are preferred for many applications.

In some embodiments, FSR (Force Sensitive Resistor) or piezo-resistive sensors or capacitive sensors may be used. One type of piezoresistive force sensor that has been used previously in footwear pressure sensing applications, known as the FLEXIFORCE® sensor, can be made in a variety of shapes and sizes, and measures resistance, which is inversely proportional to applied force. These sensors use pressure sensitive inks with silver leads terminating in pins, with the pressure sensitive area and leads sandwiched between polyester film layers. FLEXIFORCE® sensors are available from Tekscan, Inc., 307 West First Street, South Boston, Mass. 02127-1309 USA. Other types of sensors, including sensors employing conductive electrodes, may also be associated with various substrate materials (e.g., garments, sheet materials and the like). Such sensors may provide data relating to temperature, moisture, humidity, stress, strain, heart rate, respiratory rate, blood pressure, blood oxygen saturation, blood flow, local gas content, galvanic skin response, bacterial content, position, multi-axis acceleration, as well as locational positioning (GPS), and the like. A variety of such sensors are known in the art and may be adapted for use in sensing systems described herein.

In some embodiments, sensors and/or associated leads and/or conductive traces incorporated in sensing systems of the present invention comprise non-silicon-based materials such as flexible, resistive and/or conductive “e-textile” fabric and/or yarn material(s), fibers or the like. In some embodiments, sensors and/or associated leads and/or conductive traces incorporated in sensing systems of the present invention comprise flexible, resistive and/or conductive fabric or yarn materials that are substantially isotropic with respect to their flexibility and/or stretch properties. By “substantially” isotropic, we mean to include materials that have no more than a 15% variation and, in some embodiments, no more than a 10% variation in flexibility and/or stretch properties in any direction, or along any axis of the material. Suitable materials, such as resistive and/or piezoresistive and/or capacitive or conductive fabric and yarns, coated and/or impregnated fabrics and yarns, such as metallic coated fabric and yarn materials and fabric and yarn materials coated or impregnated with other types of resistive or capacitive or conductive formulations, are known in the art and a variety of such fabric and yarn materials may be used. In some embodiments, pressure sensors comprise flexible conductive woven fabric material that is stretchable and/or elastic and/or substantially isotropic with respect to its flexibility and/or stretch properties.

Fabrics and yarns comprising a knitted nylon/spandex substrate coated with a resistive or capacitive formulation are suitable for use, for example, in fabricating biometric e-textile pressure sensors and in other applications requiring environmental stability and conformability to irregular configurations. One advantage of using these types of e-textile sensors is that they perform reliably in a wide variety of environments (e.g., under different temperature and moisture conditions), and they're generally flexible, durable, washable, and comfortably worn against the skin. Suitable flexible resistive fabric and yarn materials are available, for example, from VTT/Shieldex Trading USA, 4502 Rt-31, Palmyra, N.Y. 14522, from Statex Productions & Vertriebs GmbH, Kleiner Ort 11 28357 Bremen Germany, and from Eeonyx Corp., 750 Belmont Way, Pinole, Calif. 94564.

Techniques for deriving force and/or pressure and/or shear measurements using e-textile materials are known in the art and various techniques may be suitable. See, e.g., http://www.kobakant.at/DIY/?p=913. Techniques for measuring other parameters using e-textile materials, such as humidity and temperature measurements, are also known and may be used in sensing systems of the present invention. See, e.g. http://www.nano-tera.ch/pdf/posters2012/TWIGS105.pdf. E-textile sensors of the present invention may thus be capable of monitoring various parameters, including force, pressure, shear, humidity, temperature, gas content, and the like, at the sensor site. Additional monitoring capabilities may be available using e-textile sensors as innovation in fabric sensors proceeds and as nano-materials and materials incorporating nano-structures are developed and become commercially feasible.

Flexible (and optionally stretchable or elastic) resistive and/or conductive fabric sensor(s), leads and/or traces may be associated with an underlying substrate such as fabric or sheet material that's substantially non-conductive and at least somewhat flexible. The term “fabric” or “sheet material” as used herein, refers to many types of pliable materials, including traditional fabrics comprising woven or non-woven fibers or strands, knitted substrates and materials, as well as fiber reinforced sheet materials, and other types of flexible sheeting materials composed of natural and/or synthetic materials, including flexible plastic sheeting material, pliable thermoplastic, foam and composite materials, screen-like or mesh materials, and the like. The underlying substrate may comprise a sheet material fabricated from flexible fabric material that is stretchy and/or elastic. The sheet material forming the underlying substrate may be substantially isotropic with respect to its flexibility and/or stretch properties. By “substantially” isotropic, we mean to include materials that have no more than a 15% variation and, in some embodiments, no more than a 10% variation in flexibility and/or stretch properties in any direction, or along any axis of the material. In some embodiments, the sheet material forming the carrier comprises a vinyl material; in some embodiments, the sheet material forming the carrier comprises a silicone-containing material.

Flexible resistive or conductive yarns may also be used to fabricate e-textile sensors. Such resistive or conductive yarns may be woven or knit or otherwise associated with or integrated in a substrate material according to predetermined patterns to provide a plurality of spatially distinctive sensors associated with a substrate such as a garment. In one embodiment, garments having a plurality of integrated and spatially distinct resistive or conductive sensors fabricated by weaving or knitting or otherwise associating resistive or conductive yarns or fibers in substrate materials are preferred.

In some embodiments, conductive sensors comprise other types of flexible conductive or resistive materials, such as thermoplastic elastomers (TPEs), conductive inks applied to or otherwise associated with substrate materials via printing or other process, or the like. Conductive or resistive sensors comprising materials such as TPEs, conductive inks, and the like, may be associated (directly or indirectly) with a substrate and with one or more leads or traces.

For footwear and similar applications, for example, one or more e-textile sensor(s) and/or sensing devices may be associated with (e.g., sewn or otherwise permanently or detachably attached or connected or fixed to, or woven or knit or integrated in) a footwear component, such as an inner, intermediate or outer layer for contacting an individual's foot, directly or indirectly, during use. Conductive sensors may be used, for example, to detect electrical impulses for monitoring vital signs, skin conductance, or the like; resistive or capacitive sensors may be used, for example, for detecting pressure and/or force and/or shear exerted against an individual's skin or other force-related parameters sensed at or near a skin surface.

Each sensor may be associated with one or more leads, each of the leads being electrically connected to a conductive trace conveying electrical signals from the sensor to a signal transfer terminal. In some embodiments, e-textile sensors as previously described may be electrically connected to lead(s), or e-textile sensors may have flexible yarn or textile lead(s) associated with or incorporated in the e-textile sensor footprint. The lead(s) are electrically connected to flexible conductive traces, which may comprise a variety of flexible conductive materials, such as a conductive fabric, yarn, fibers or the like. In some embodiments, the conductive traces are stretchable and/or elastic, and are woven or knit into and form part of the substrate.

In some embodiments, conductive traces comprise a conductive e-textile fabric having generally high electrical conductivity, such as silver coated e-textile materials, and may be bonded to the underlying substrate material using adhesives, heat bonding or non-conductive threads. Suitable e-textile materials are known in the art and are available, for example, from the vendors identified above. In some embodiments, conductive traces comprise a conductive yarn or fiber having generally high electrical conductivity, and the yarn or fiber materials are integrated into the substrate material by knitting, weaving, or the like. In some embodiments, the conductive traces comprise a conductive yarn or fiber having generally high electrical conductivity, and having an insulative coating, and the insulated, conductive yarn or fiber materials are integrated into the substrate material by knitting, weaving, or the like. In some embodiments, conductive traces comprise other types of flexible conductive materials, such as thermoplastic elastomers (TPEs), conductive inks applied to or otherwise associated with the substrate via printing or other process, or the like. Conductive traces comprising materials such as TPEs, conductive inks, and the like, may be associated (directly or indirectly) with a substrate and with one or more leads or sensors to provide conductive pathways between the sensors and corresponding signal transfer terminals.

Each of the conductive traces terminates in a signal transfer terminal that is mounted to/in/on, or otherwise associated with (referred to, collectively, as “associated with”), the underlying substrate (e.g., footwear) and can be contacted to a mating signal receipt terminal of a dedicated electronic device (DED) having data storage, processing and/or analysis capabilities. In general, conductive traces and terminals are arranged in a predetermined arrangement that corresponds to the arrangement of signal receipt terminals in the DED. Many different types of signal transfer and receipt terminals are known and may be used in this application. In one exemplary embodiment, signal transfer and receipt terminals may be mounted in cooperating fixtures for sliding engagement of the fixtures and terminals. In another embodiment, signal transfer terminals may be provided as conductive fixtures that are electrically connected to the conductive trace (and thereby to a corresponding sensor) and detachably connectible to a mating conductive fixture located on the DED.

In some embodiments, the mating signal receipt and signal transfer terminals may comprise mechanically mating, electrically conductive members such as snaps or other types of fasteners providing secure mechanical mating and high integrity, high reliability transfer of signals and/or data. In some embodiments, the mating terminals may comprise conductive pins, including stationary conductive pins as well as movable pins, such as spring-loaded pins, referred to as pogo pin connectors. In some embodiments, easy and secure mating of the terminals may be enhanced using magnetic mechanisms or other types of mechanisms that help users to properly and securely align and connect/disconnect the mating terminals with minimal effort. In some embodiments, easy and secure mating of the terminals may be enhanced by complementary (and/or locking) mechanical configurations of housing components associated with mating terminals. In some embodiments, mating terminals provided on the underlying substrate (e.g., a garment, sock, sheet, band, etc.) and on a DED are provided in a predetermined arrangement, or have a keyed configuration, to ensure that the DED is properly aligned and mounted to the terminals provided on the substrate in a predictable and pre-determined orientation.

The DED, in addition to having data recording, processing and/or analysis capabilities, may incorporate an energy source such as a battery providing energy for data recording, processing and/or analysis, as well as providing energy for operation of one or more of the sensor(s). The energy source may comprise a rechargeable and/or replaceable battery source, and/or a regenerative energy system. The DED generally provides a lightweight and water-tight enclosure for the data collection and processing electronics and (optional) energy source and provides signal receiving terminals that mate with the signal transfer terminals connected to the sensor(s) for conveying data from the sensors to the dedicated electronic device. In some embodiments, the DED is provided as a bendable or partially bendable device that can be shaped, as desired, to fit comfortably on and closely to body surfaces having different configurations and sizes.

A DED may be provided in the form of a curved band for mounting to the user at or near the user's ankle, and particularly at or near a front-facing portion of the user's ankle, for example, and may be at least partially flexible so that it fits, comfortably and functionally, on men's and women's ankles and on ankles having different sizes and shapes, providing connection to the sensor transfer terminals provided in a sock or anklet incorporated in footwear. In some embodiments, a partially or fully bendable DED may be used in a variety of configurations, including, e.g., flat or substantially flat configurations, depending on the location of sensor transfer terminals provided in an underlying substrate. In some embodiments, a partially or fully bendable DED may be used in different configurations with sensor transfer terminals provided in different form factors. For example, a common DED may be shaped to fit comfortably on a user's ankle and mate with sensor transfer terminals provided on an underlying sock or anklet; it may also be shaped to fit comfortably on a user's arm and/or wrist and mate with sensor transfer terminals provided on an underlying sleeve. The same bendable DED may additionally be shaped to fit comfortably, in a generally curved or a generally flat configuration, and mate with sensor transfer terminals provided on garments or substrates having other form factors.

DEDs having alternative configurations are also disclosed and may be used in a variety of applications. In some embodiments, a DED may be provided in the form of a button-like or dongle-like or capsule-like object having signal receipt terminals that mate with signal transfer terminals provided in a mating DED-receiving fixture that may be mounted to or incorporated in (referred to, collectively, as “associated with”) footwear or another substrate. In some embodiments, the DED-receiving fixture may comprise a substantially flexible and bendable material and may be mounted to a footwear substrate at a heel portion, a plantar sole portion, or at another site associated with footwear. In some embodiments, multiple DED-receiving fixtures and DEDs may be used for various monitoring and data collection purposes.

In some embodiments, the DED communicates with and transfers data to one or more external computing and/or display system(s), such as a smartphone, computer, tablet computer, dedicated computing device, base station (hub), medical records system or the like, using wired and/or wireless data communication means and protocols. The DED and/or an external computing and/or display system may, in turn, communicate with a centralized host computing system (located, e.g., in the Cloud), where further data processing and analysis takes place.

In some embodiments, the DED might advertise its presence to other DEDs, initiate discovery of other DEDs found in proximity, respond to advertising of other DEDs, and otherwise negotiate with all the participating DEDs the proper protocol to exchange data with the purpose of transmitting their data at longer range, for example, through a “mesh network”. A mesh network has a topology whereby all devices can communicate with all other devices in the network, either directly if in range, or indirectly via one or more intermediate “nodes” if they are not. This is in contrast to other network types that often feature a central hub like a router, through which all traffic must flow. Mesh networks have no such central hub and offer multiple ways of getting data from one device to another.

Substantially real-time feedback, including data displays, notifications, alerts and the like, may be provided to the user, caretaker and/or clinician according to user, caretaker and/or clinician preferences. In some embodiments, the DED may store data temporarily to a local memory, and may periodically transfer the data (e.g., in batches) to the above mentioned external computing and/or display system(s). Offline processing and feedback, including data displays, notifications and the like may be provided to the user, caretaker, and/or clinician according to user, caretaker and/or clinician preferences.

In operation, an authentication routine and/or user identification system matches the DED and associated sensing system (e.g., the collection of sensor(s) associated with an underlying substrate) with the user, caretaker and/or clinician, and may link user information or data from other sources to a software- and/or firmware-implemented system residing on the external computing system. The external computing device may itself communicate with a centralized host computing system or facility where data is stored, processed, analyzed, and the like, and where output, communications, instructions, commands, and the like may be formulated for delivery back to the user, caretaker and/or clinician through the external computing device and/or the DED.

Calibration routines may be provided to ensure that the DED and connected related sensor system are properly configured to work optimally for the specific user. Configuration and setup routines may be provided to guide the user (or caretaker or medical professional) to input user information or data to facilitate data collection, and various protocols, routines, data analysis and/or display characteristics, and the like, may be selected by the user (or caretaker or medical professional) to provide data collection and analysis that is targeted to specific users. Specific examples are provided below. Notification and alarm systems may be provided, and selectively enabled, to provide messages, warnings, alarms, and the like to the user, and/or to caretakers and/or medical providers, substantially in real-time, based on sensed data.

Various other aspects of sensing systems and background relating to the construction, use and utility for such sensing systems are described in the following previously published and commonly owned patent publications, all of which are incorporated herein by reference in their entireties: U.S. Pat. No. 8,925,392; PCT Patent Publication 2013/116242 A2; PCT Patent Publication 2015/017712 A1; U.S. Patent Publication US-2015-0182843-A1; and PCT Patent Publication WO 2015/175838 A1.

### BRIEF DESCRIPTION OF THE DRAWINGS

FIGS. 1, 2 and 3 show images illustrating a plurality of sensors and associated conductive traces associated with a footwear upper component according to one embodiment of footwear disclosed herein.

FIG. 4 shows an image illustrating a heel portion of one embodiment of a footwear component illustrating conductive traces terminating at trace terminations and conductive bridges.

FIG. 5 shows an image illustrating the interface of footwear outer and inner sock components at the location of conductive traces in the heel region according to one embodiment of footwear disclosed herein.

FIGS. 6 and 7 show images illustrating one embodiment of a DED and associated mounting band provided at a heel region of footwear according to one embodiment of footwear disclosed herein.

FIG. 8 shows an image illustrating another embodiment of a DED and associated mounting band provided at a heel region of footwear according to one embodiment of footwear disclosed herein.

FIG. 9 shows images illustrating additional embodiments of DEDs provided at alternative footwear locations according to alternative embodiments of footwear disclosed herein.

FIGS. 10A-10C illustrate another embodiment of an exemplary tab having signal receipt terminals arranged on a tab portion and signal transfer terminals arranged in a DED receipt cavity. FIG. 10A shows an upper perspective view of the exemplary tab having a tab portion and a DED receiving fixture; FIG. 10B shows a lower perspective view of the exemplary tab illustrating signal receipt terminals provided on the bottom of the tab portion; and FIG. 10C shows a perspective view, with portions of the structure shown as transparent, illustrating the signal receipt terminals provided on the lower surface of the tab portion and signal transfer terminals provided in the DED receiving fixture. In the embodiments illustrated, the tab portion is symmetrically formed, with the right and left sides having the same appearance; likewise, both the inner and outer conformations of the DED receiving fixture are symmetrically formed, with right and left sides having the same appearance.

FIGS. 11A and 11B illustrate another embodiment of a button-like DED sized and configured for receipt in a DED receipt cavity of a tab as illustrated in FIGS. 10A-10C. FIG. 11A shows an upper perspective view of the DED, showing the external surface with an underlying interface portion, and FIG. 11B shows a lower perspective view of the DED, showing the interface portion having a plurality of signal receipt terminals extending from an internally-directed surface. In the embodiments illustrated, the DED is symmetrically formed, with right and left sides having the same appearance.

FIGS. 12A-12D illustrate a DED as illustrated in FIGS. 11A and 11B mounted in the DED receipt cavity of the exemplary tab of FIGS. 10A-10C. FIGS. 12A and 12B show plan and perspective views of the external configurations of the interfacing DED and tab components; FIG. 12C shows a top perspective view of the DED and tab components, with portions of the structure shown as transparent, illustrating the location of signal receipt terminals located on a bottom surface of the tab; and FIG. 12D shows a side view of the DED and tab components illustrating the location of signal receipt terminals on a bottom surface of the tab and signal transfer terminals located in a DED receipt cavity of the tab. In the embodiments illustrated in FIGS. 12A-12D, the external configurations of the interfacing DED and tab components are symmetrically formed, with right- and left-sides having the same configurations.

FIGS. 13A and 13B show highly schematic views of the DED and tab combination illustrated in FIGS. 10A-12D associated with a sock substrate.

FIGS. 14A-14C illustrate top perspective, bottom perspective and upper side perspective views, respectively of a charging station for a DED as illustrated in FIGS. 11A and 11B. FIG. 14A shows an upper perspective view of the external configuration of a charging station having a DED receipt cavity for mating with a DED as illustrated in FIGS. 11A and 11B. FIG. 14B shows a lower perspective view of the external configuration of a charging station of FIG. 14A. FIG. 14C illustrates an upper perspective view of the external configuration of a charging station as shown in FIG. 14A, illustrating charging pins in electrical communication with a charging interface. In the embodiment illustrated in FIGS. 14A-14C, the external configuration of the charging station is symmetrically formed, with right- and left-hand sides having the same configuration.

FIG. 15 illustrates an exemplary report mapping various parameters relating to an individual's performance measured using sensor-enabled footwear as described herein.

FIG. 16 illustrates another exemplary report mapping various parameters relating to a team's performance measured using sensor-enabled footwear as described herein.

It will be understood that the appended drawings are not necessarily to scale, and that they present one embodiment of many aspects of systems and components of the present invention. Specific design features, including dimensions, orientations, locations and configurations of various illustrated components may be modified, for example, for use in various intended applications and environments.

### DETAILED DESCRIPTION OF EXEMPLARY EMBODIMENTS

In one embodiment, systems incorporating sensors, traces and terminals may be associated with footwear. Although a specific embodiment of sensing systems is illustrated and described with reference to specific types of sensors, traces, bands, conductive bridges and terminals associated with a footwear substrate (e.g., shoes, specialized sports shoes, insoles, boots, etc.), it will be appreciated that similar fabrication techniques and features may be used in connection with a variety of sensors, traces, terminals and substrates, including various types of garments (e.g. socks, shorts, t-shirts and jerseys), belts, straps, sporting equipment (e.g., shin guards and other protective gear), and the like. The term “sensor,” as we use it herein, refers to the various types of sensors as described herein, as well as additional means for sensing as that term may be construed to extend to sensors as described herein as well as other, additional types of sensors that may be associated with sensing systems as described.

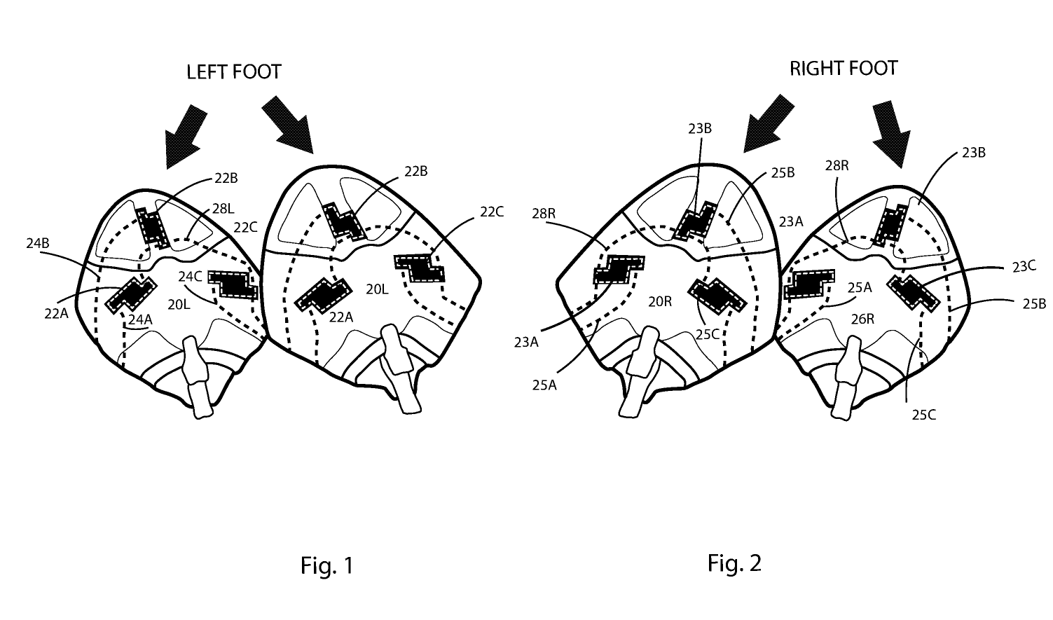

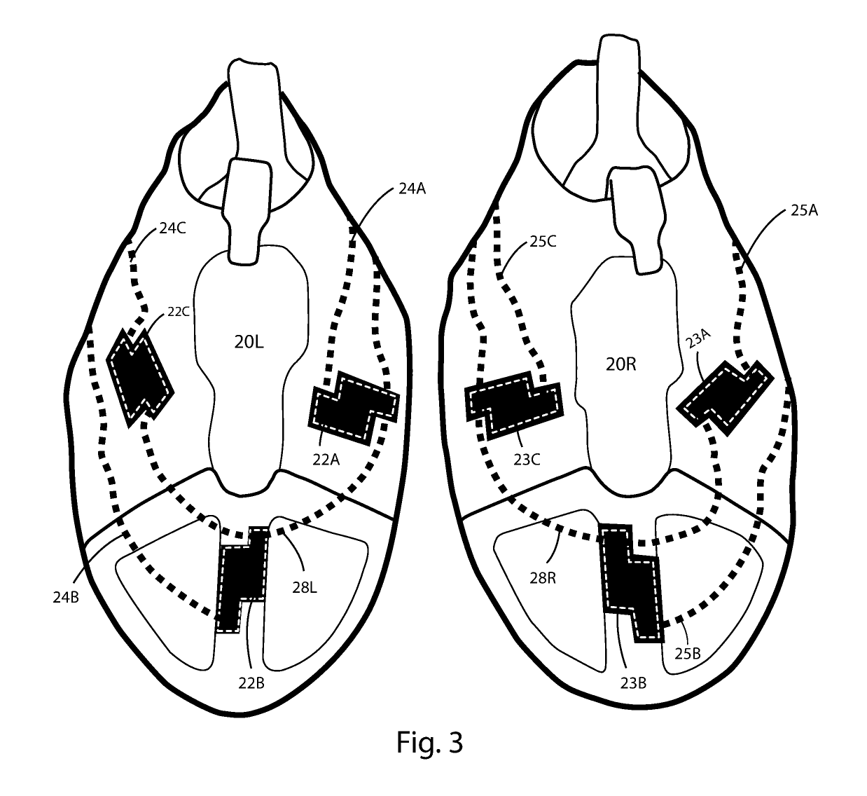

FIGS. 1-3 illustrate substrates in the form of footwear inners comprising a substrate material equipped with a plurality of sensors, leads, and traces providing signals and/or data to a dedicated (and optionally detachable) electronic device that gathers data from each sensor and communicates with an external display, computing and/or mobile device. In this exemplary embodiment, sensors used in footwear and/or sock applications may comprise e-textile pressure sensors capable of detecting levels of pressure (and/or force and/or shear or derivative measurements) at one or more areas of the foot and may include other types of sensors, including electrically conductive electrodes, vital sign monitoring sensors, accelerometers, gyroscopes, electromyography sensors, moisture sensors, and the like.

The embodiments illustrated in FIGS. 1-3 show footwear inner components such as inner components 20L, 20R (left and right, respectively) having a plurality of pressure sensors 22A-C, 23A-C provided in locations corresponding to dorsal and/or lateral areas of the left and right foot, respectively, when worn. In these embodiments, one pressure sensor 22B, 23B is located at a central dorsal portion of the left and right foot, respectively, overlying a central dorsal toe region of the foot; and one or more additional pressure sensor(s) 22A, 23A, 22C, 23C may be located laterally of the central dorsal portion of the foot, on either side of the foot. This sensor arrangement is useful for monitoring pressure exerted on the dorsal area of the foot, such as by a ball, and is especially suitable for use with soccer footwear. Providing pressure sensors in central and lateral dorsal toe and/or forefoot locations allows monitoring of ball touches in various regions of each foot, as well as providing data relating to the force of various ball touches. In additional or alternative embodiments, pressure sensors may be provided at additional or alternative locations corresponding to a foot, including plantar regions (e.g., plantar forefoot, heel, midfoot, arch, etc.), heel regions, ankle regions, and additional dorsal and/or lateral regions of the foot.

For the illustrated embodiments incorporating pressure sensors, parameters such as pressure, force and/or shear are detected at one or more areas of the foot, and trends in those parameters detected over one or more monitoring period(s) may produce conclusions relating to the user's performance and interaction with a ball, the user's gait, walking or running style, cadence, foot landing, susceptibility to injury, etc. can be drawn and feedback can be provided to the user, and/or to a third party (e.g., coach, care provider, physical therapist, group, etc.) to report activity, progression, susceptibility to injury, or the like. In addition, notifications, alerts, recommended actions, and the like may also be communicated to the user, caretaker and/or clinician based on the data analysis, essentially in real time. These systems are suitable for use in many different types of applications.

In the illustrative embodiment illustrated in FIGS. 1-3, pressure sensor locations on each of a pair of shoe inner components (right and left) are located in mirror-image locations, with two pressure sensor locations on each footwear component being located on opposite sides of the dorsal forefoot or toe area and one pressure sensor location at a generally central dorsal forefoot or toe area. In the embodiment illustrated and described in more detail below, e-textile fabric pressure sensors are associated with (e.g., fastened to, sewn to, adhered to, knit or woven into, or the like) an internal or intermediate footwear component (e.g., footwear inner or intermediate layer) at the designated sensor location(s) intermediate the exterior footwear surface and the user's foot when the footwear is worn. It will be appreciated that sensor placement may vary, that different types of sensors may be implemented, that additional sensors may be incorporated in sensing systems (e.g., footwear) as described herein.

The substrate material 20 associated with sensor(s) is generally pliable and may be stretchable, and it is substantially non-electrically conductive. Natural and synthetic materials that are known and used in fabricating footwear components are suitable. In some embodiments, sensors are associated with an exterior surface of a mesh-like insert or liner positioned in a footwear upper. It will be appreciated that sensors may additionally or alternatively be associated with an interior surface of an insert, liner or footwear upper, or with exterior surfaces of a footwear upper, and that additional intermediate layers or materials may be provided.

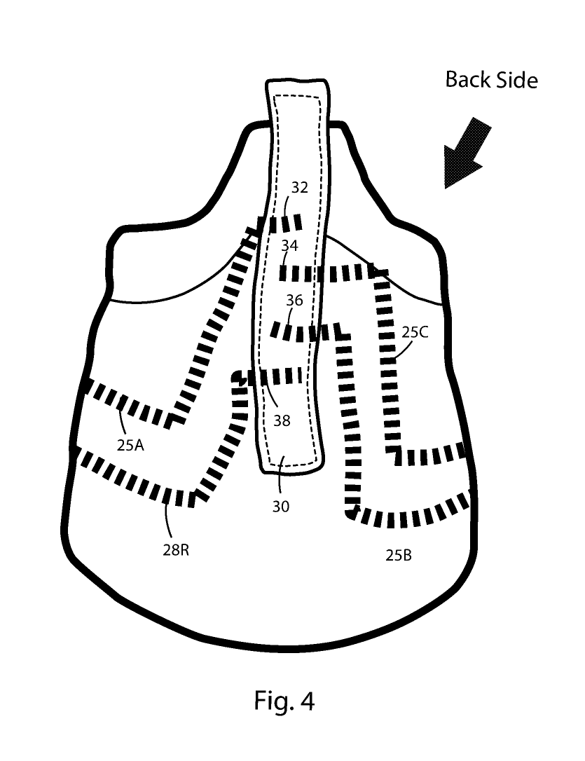

The dashed lines and pathways shown in FIGS. 1-3 leading from sensors locations illustrate electrically conductive traces fabricated from electrically conductive yarns, thread, material, or the like, which may be applied or fastened or adhered to the substrate, or knit or woven or stitched into the substrate, as shown. In some embodiments, the electrically conductive traces may include an insulative cover or outer layer to provide more stable electrical signal pathways. In some embodiments, the electrically conductive traces may be formed, for example, using conductive thermopolymer elastomers (TPEs), conductive inks, and the like. The conductive traces 24A-C, 25A-C provide electrical pathways connecting sensors 22A-C, 23A-C or sensor leads to signal transfer locations, which are illustrated in FIG. 4 as trace terminations or conductive bridges 32, 34, 36, 38 located at a heel region 30 of the footwear and, in the illustrated embodiment, oriented horizontally.

In some embodiments, one or more common or ground traces provide electrical ground pathways contacting each sensor or sensor lead location and terminating at one or more trace terminations or conductive bridges. In the embodiments illustrated in FIGS. 1-4, a common or ground trace 28L, 28R contacts each sensor (and/or sensor lead), serially, and terminates at a common or ground trace termination or conductive bridge. The four trace terminations shown at the heel region in FIG. 4 correspond to three sensor signal trace terminations 32, 34, 36 (one for each sensor) and one common or ground trace termination 38 that contacts each sensor.

The conductive traces shown in FIGS. 1-4 comprise electrically conductive yarn or thread or other material providing an electrically conductive pathway between each of the sensors or sensor leads and a location in proximity to signal transfer terminals. Electrically conductive materials available under the mark X-STATIC are suitable for fabricating conductive traces as described herein, and insulated conductive materials may also be employed. In the embodiments illustrated, the traces are integrated with the substrate material by stitching. The length and width of conductive trace pathways may be modified and adjusted for different applications, depending on the impedance properties of the sensing system and electronics. In some embodiments, the width of one or more trace pathways formed by the conductive yarn or thread may vary over the length of the trace pathway.

The sensor and ground trace terminations are shown more clearly in FIG. 4. Each trace, including sensor traces and ground trace(s) terminate at an area spaced from the trace terminations. In some embodiments, conductive bridges are provided at trace termination locations as horizontally oriented areas of densely stitched conductive thread to facilitate electrical contact between a terminal area of each trace and a corresponding conductive terminal. Conductive bridges provide highly conductive contact areas that may be elevated with respect to the surface of the underlying substrate to facilitate stable and reliable electrical contact. In some embodiments, conductive bridges associated with the substrate may be stiffer, or less flexible, than the underlying substrate material. In some embodiments, a conductive bridge may be provided corresponding to each terminal area of each trace; in some embodiments, conductive bridges are provided for more or fewer than each of the terminal areas of each trace. Conductive bridges having configurations other than rectangular and orientations other than horizontally directed may be provided. In some embodiments, conductive bridges provided at different trace termination locations may have different configurations, orientations, and the like.

Conductive bridges may be arranged in a generally aligned arrangement as shown in FIG. 4 and, in some embodiments, each conductive bridge is separated from neighboring bridges by a distance of at least about 5 mm and, in some embodiments, by a distance of about 1 cm. In some embodiments, conductive bridges are separated from neighboring bridges by a distance of no less than 7 mm and no more than 1.5 cm and, in some embodiments, by a distance of no less than 7 mm and no more than 1.2 cm. The conductive threads forming conductive bridges may be densely stitched in an orientation substantially transverse to the major longitudinal dimension of the formed bridge. As a result of the dense pattern of conductive material, the conductive bridges provide a substantially continuous, electrically conductive surface layer that is raised relative to the surface of the knit substrate in that area. The bridges provide a contact surface or “pad” providing a consistent and stable conductive surface for electrically contacting a “rear” face of signal transfer contacts or conductive terminals. In alternative embodiments, conductive bridges comprise other types of conductive materials associated with the substrate, or with a terminal mounting band. Conductive bridges may be formed, for example, using conductive thermopolymer elastomers (TPEs), conductive inks, and the like.

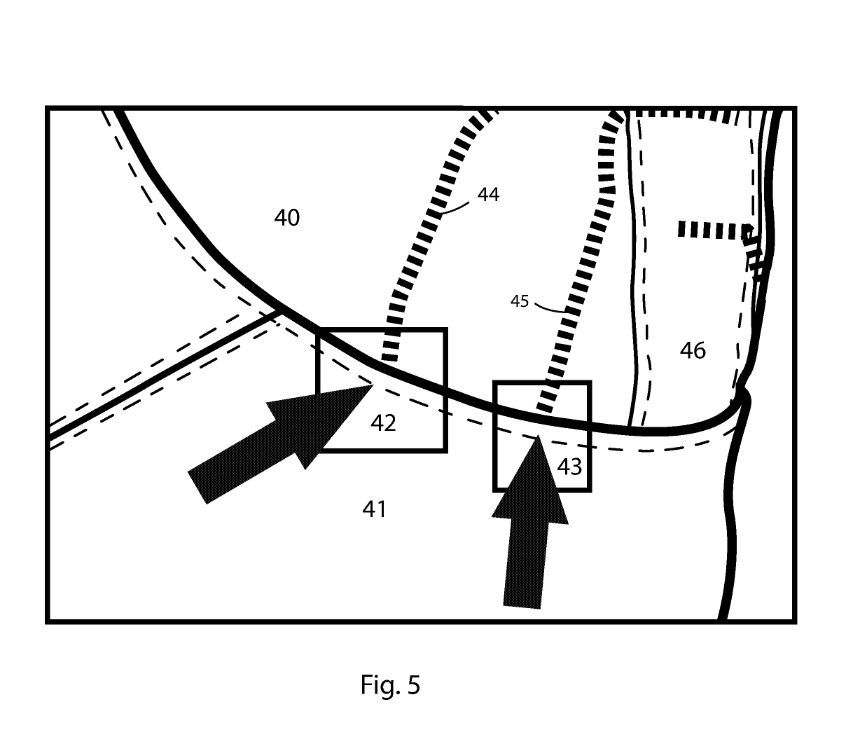

FIG. 5 illustrates one embodiment in which a substrate such as an inner or liner incorporates one or more sensors 42, 43 and conductive traces 44, 45 associated with a sock-like substrate 40 extending from and above a shoe outer in the heel and ankle region of the footwear. Conductive traces traverse the sock-like substrate 40 and terminate at trace terminations located on a non-woven and non-knit heel tab 46.

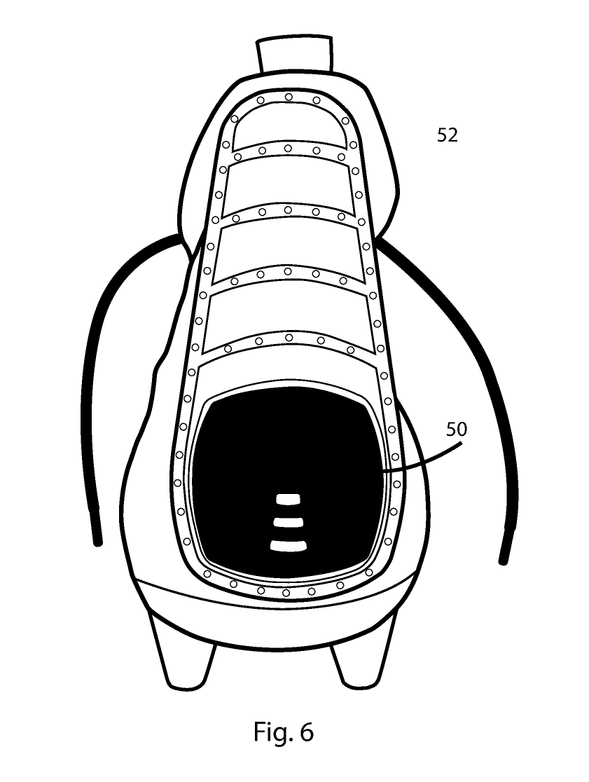

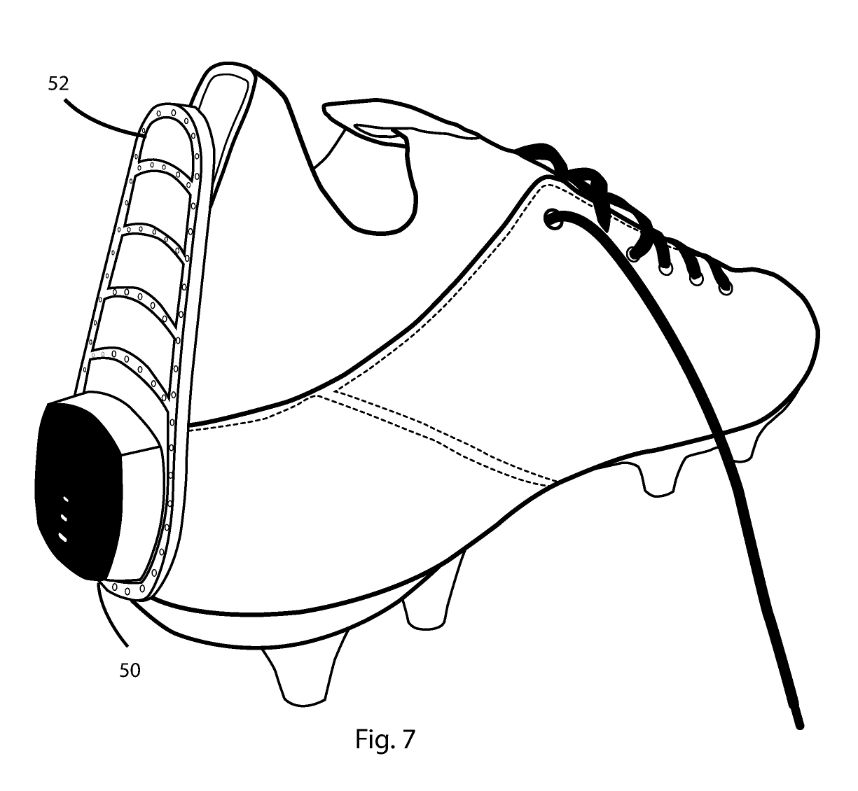

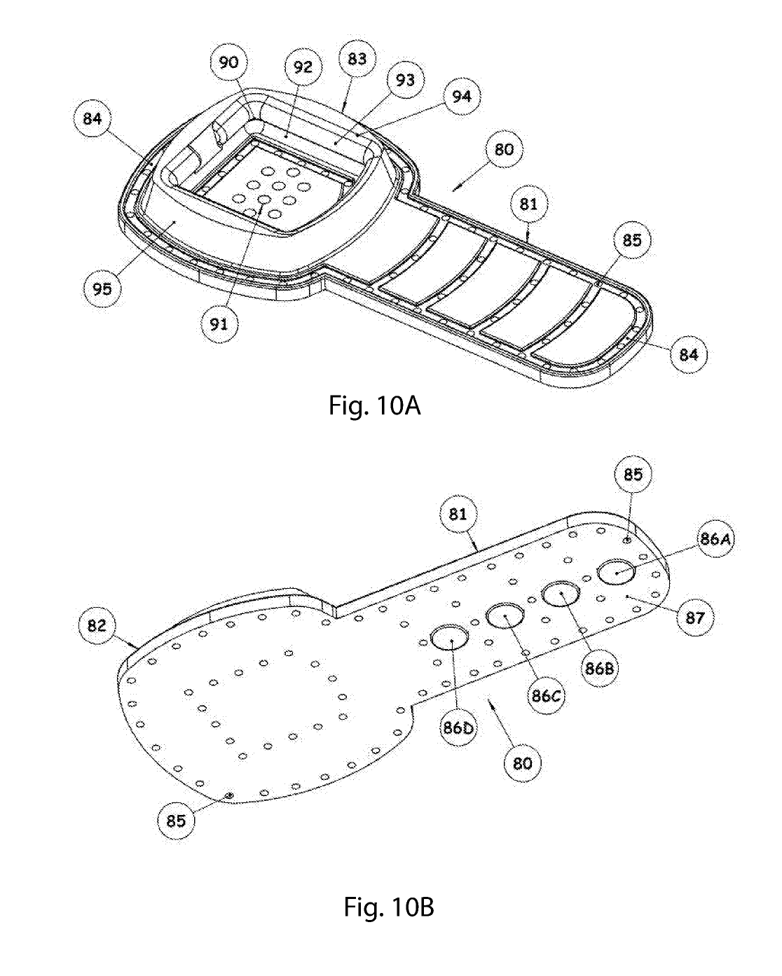

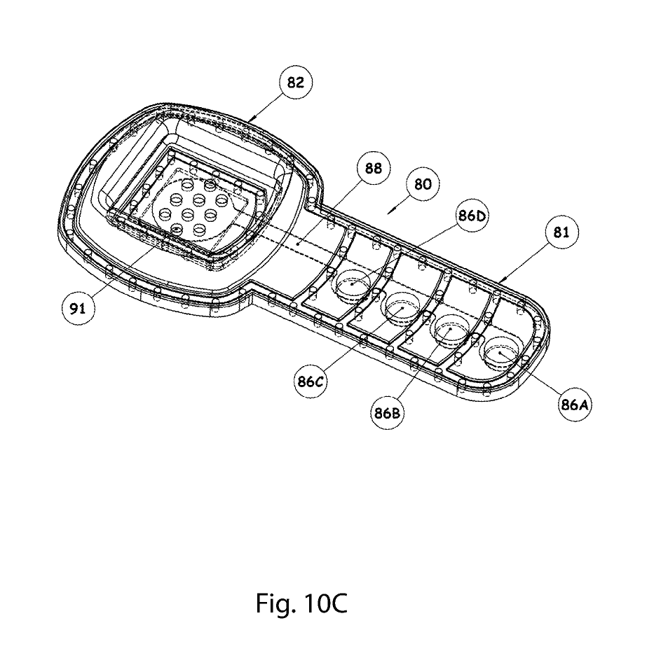

In some embodiments, illustrated in FIGS. 6 and 7, a dedicated electronic device (DED) 50, illustrated as the dark button-like object, is mounted, detachably or semi-permanently or permanently, at the heel region of the footwear. In the footwear embodiments illustrated in FIGS. 6 and 7, trace terminations and/or conductive bridges communicating with one or more sensors are located underneath a heel tab portion 52, which is shown as a vertically oriented, non-conductive tab. Trace terminations and/or conductive bridges such as those shown in FIG. 4 are in electrical contact with corresponding contacts located within heel tab portion 52. The interior contacts may be in electrical contact with signal transfer terminals located within DED 50. Mounting tabs and DEDs having configurations and functions suitable for use in footwear applications, as described, are shown in FIGS. 10A-12D.

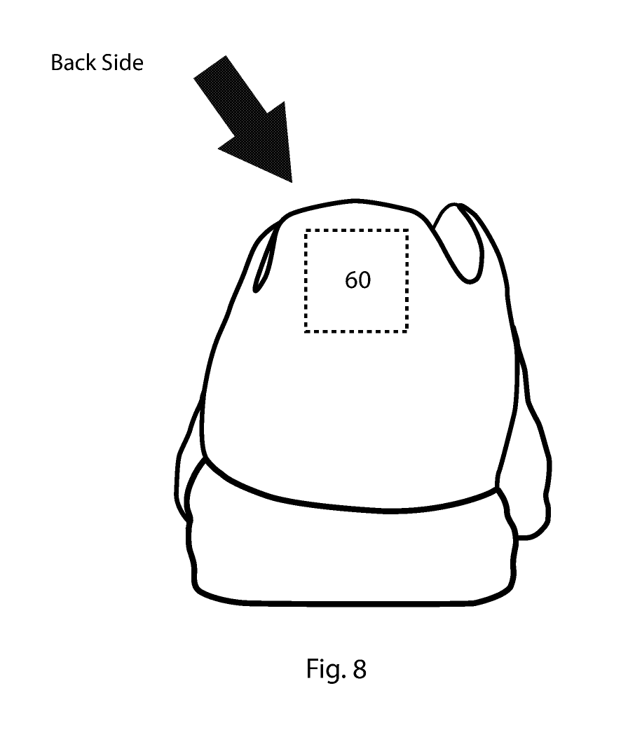

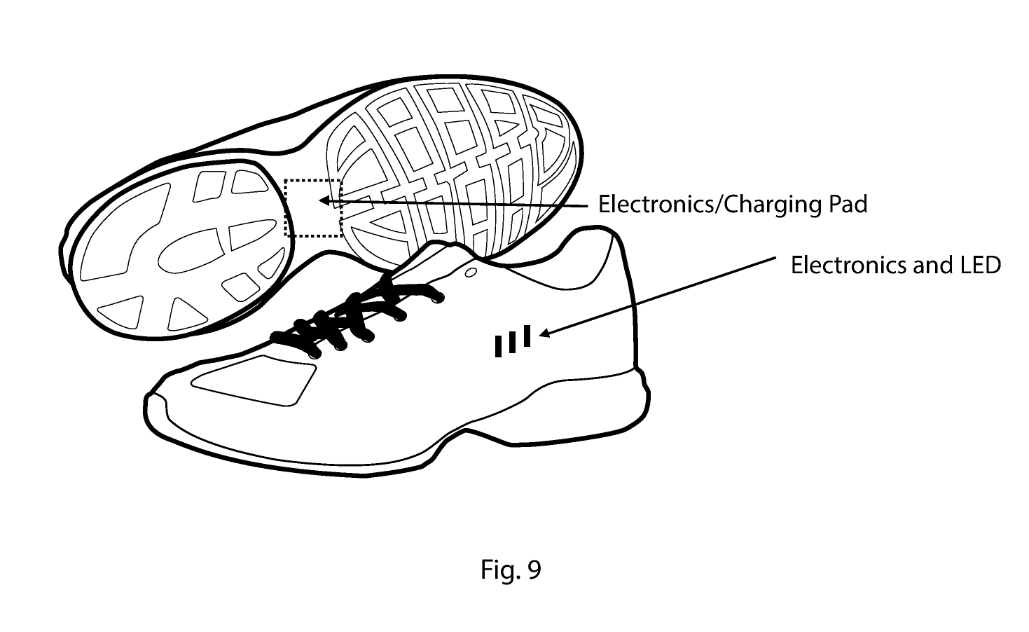

FIGS. 8 and 9 show images of footwear illustrating different DED placement. In the shoe shown in FIG. 8, for example, the DED 60 may be located in an upper and outer heel area, housing signal transfer terminals that interface with trace terminations and/or conductive bridges located between the DED and the footwear sole. FIG. 9 shows one shoe embodiment (upper shoe) in which the DED is located on or in proximity to the sole of the footwear, and another shoe embodiment (lower shoe) in which the DED is located on a side of the shoe upper (at the three vertically aligned lines). DEDs may be located at other footwear locations as well.

FIGS. 10A-12D illustrate an embodiment of a mounting tab and DED suitable for use with footwear as described herein for receiving signals from sensors and signal transfer terminals located on an underlying substrate. FIGS. 10A-10C show a mounting tab 80 having a band portion 81 and a DED receiving portion 82 including a DED receiving cavity 90 having a plurality of contacts 91 for mating with complementary contacts on a mating DED. Band and DED receiving portions 81, 82, respectively, may be associated with an underlying substrate (such as a garment) using a variety of mounting and attachment means, such as adhesives, material fusing, various types of fasteners, stitching, and the like. In the embodiment illustrated in FIGS. 10A-10C, grooves 84 having bores 85 are provided on an exterior (upper) surface along peripheral regions of band portion 81 and DED receiving portion 82 of mounting tab 80 to facilitate stitching of the mounting band to an underlying substrate. Additional grooves and bores may be provided traversing band portion 81 and along a bottom wall of DED receiving cavity 90, as shown.

A plurality of contacts (illustrated as contacts 86A, 86B, 86C, 86D) are provided penetrating and projecting from an interior (lower) surface 87 of band portion 81 of mounting tab 80. Contacts 86A-86D are configured and aligned for interfacing with and electrically contacting trace terminal portions or conductive bridges provided on an underlying substrate. The size, configuration, alignment and number of mounting tab contacts may vary depending on the size, configuration, alignment and number of trace terminations provided on a substrate.

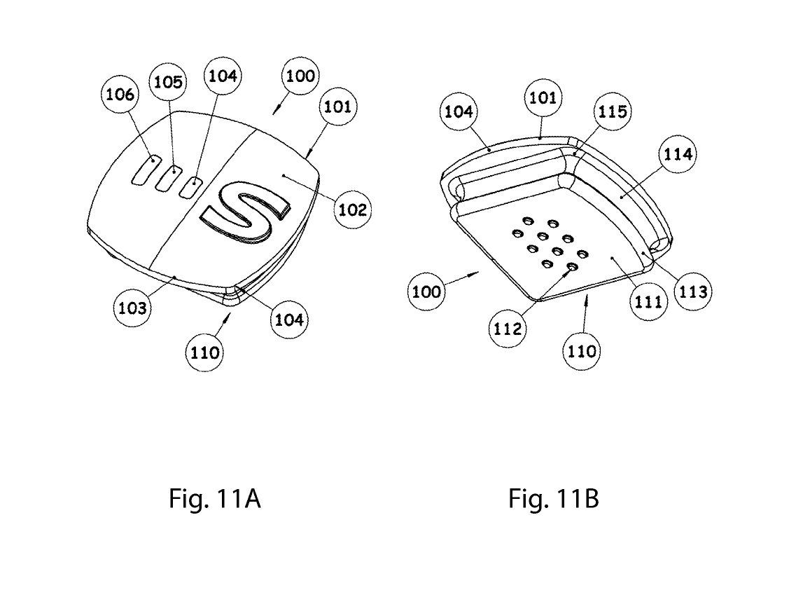

Contacts, illustrated as contacts 86A-D, are electrically connected to multiple electrically conductive pins 91 positioned in a contact interface region via electrical pathway 88, illustrated in FIG. 10C. The underside or interior surface of conductive pins 91 is visible in the view shown in FIG. 10C; the upper and DED interface surface of conductive pins 91 is visible in the view shown in FIG. 10A. The exposed interface surfaces of conductive pins 91 are located on an exposed surface of DED receipt cavity 90—on a lower, internal surface in the embodiment shown in FIG. 10A. The upper and side wall contours of DED receipt cavity 90 correspond generally to the outer contours of DED body, as shown in FIGS. 11A, 11B, to provide detachable yet stable mounting of DED body portion 110 within DED receipt cavity 90. Exterior surfaces of DED receipt cavity 90 may be provided as tapered external side walls 95.

In the specific embodiments illustrated in FIGS. 10A-11B, DED 100 comprises an exterior surface member 101 and an internal DED body 110 having an exterior configuration that mates with mounting tab DED receiving cavity 90. Exterior surface member 101 comprises an exterior surface 102 that may be smooth or contoured, and may have raised decorative or marketing indicators, system status indicators, or the like. Exterior surface member 101 has a perimeter wall 103 having a rounded polygonal configuration and a peripheral rim 104. Exterior surface member 101 may display optional indicators such as indicators 105, 106, 107 (e.g., LEDs) for communicating various system operational conditions, charge status, operational status, and the like. Exterior surface member 101 may carry additional or different user interface features, actuators, displays, decorative matter, and the like. Exterior surface member 101, as illustrated, has a perimeter larger than that of internal DED body 110.

Internal DED body 110, as shown in FIGS. 11A, 11B, comprises an internal surface 111 and a plurality of conductive pins 112 exposed on and/or projecting from internal surface 111. Conductive pins 112 may be provided as spring-loaded conductive pins, often referred to as pogo pins, to facilitate reliable contact with contacts 91 in the DED receiving cavity 90. Side walls of internal DED body 110 have a contoured configuration that is complementary to the contoured configuration of DED receipt cavity 90, facilitating convenient, stable and detachable positioning of DED 100 within DED receipt cavity 90. In the specific embodiment illustrated, internal DED body 110 comprises an internal rim 113, an intermediate groove 114 and an interface edge 115, each contoured surface being sized and configured for mating with complementary features of the DED receiving cavity 90, including internal channel 92, lip 93 and interface surface 94, respectively.

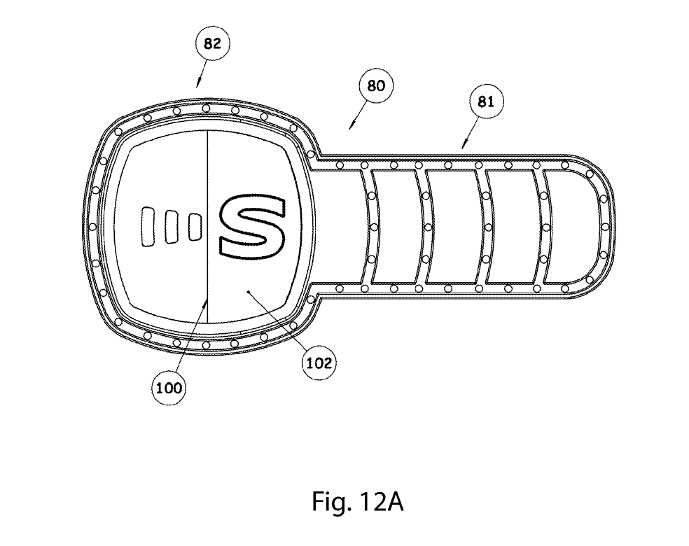

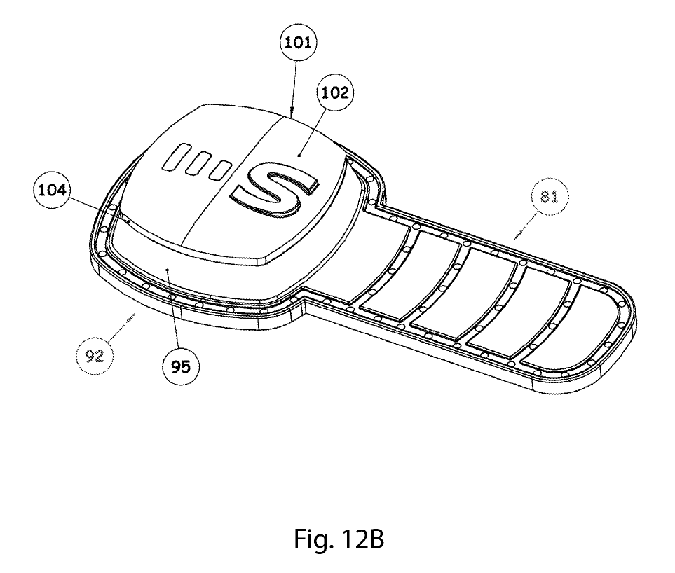

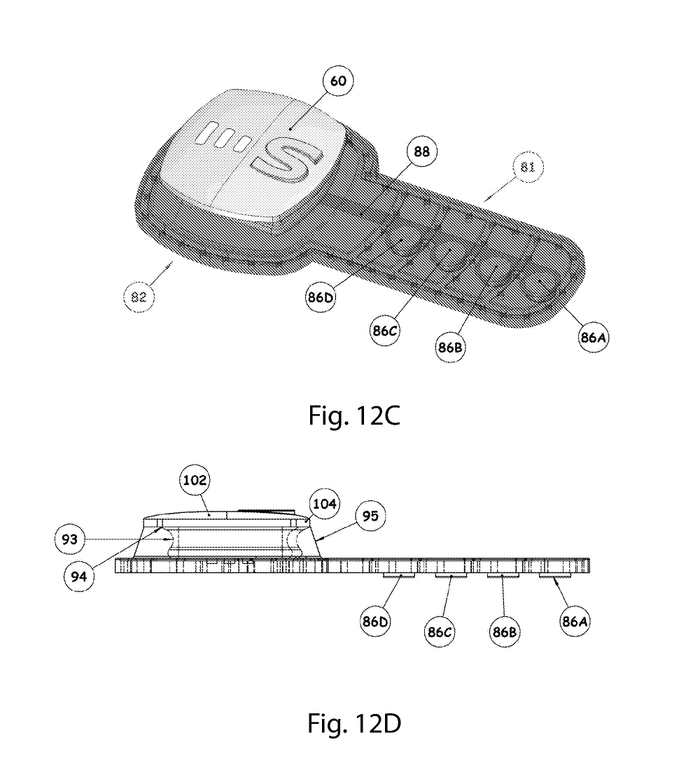

FIGS. 12A-12D show DED 100 mounted in mounting tab 80. Internal DED body 110 is enclosed within DED receipt cavity 90 in a substantially sealed manner. Tapered external side walls 95 of the DED receiving portion 82 and rim 104 are sized and configured to align and substantially seal DED receipt cavity 90. While DED 100, internal DED body 110 and DED receipt cavity 90 are illustrated having a generally square perimeter, it will be appreciated that other configurations may be used, including circular, oblong, other polygonal configurations, and other curved configurations.

Mounting tab 80 is generally constructed from a flexible, bendable non-conductive material such as a non-conductive, flexible thermoplastic elastomer (TPE), silicone, or the like. DED 100 is generally constructed from a harder, more rigid material, and may house electrical and electronic components such as one or more accelerometer(s); one or more gyroscope(s); one or more magnetometer(s); one or more 6-axis and/or 9-axis inertial measurement units IMU(s); data processing; data storage (e.g., flash memory); data communications (e.g., Bluetooth, ANT+, Wi-Fi; and/or Proprietary TX/RX protocols); energy source(s) (e.g., rechargeable battery/ies, energy harvesting modules, and the like); antenna/e for wireless communications; and a plurality of analog sensor inputs (for pressure, temperature, humidity, and other sensor parameters).

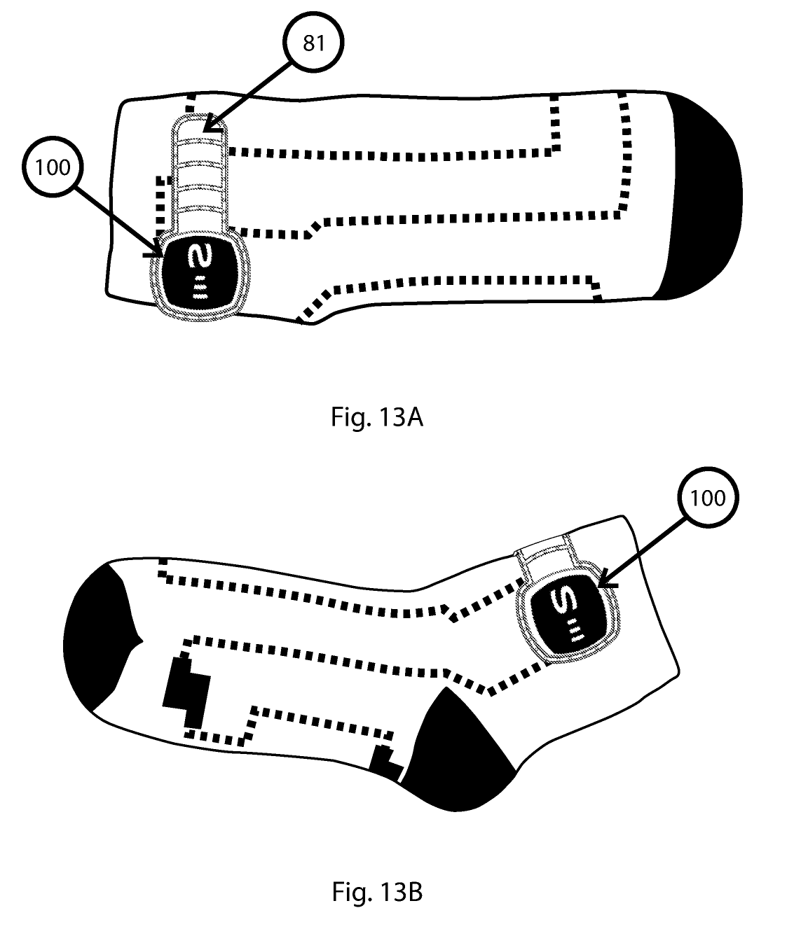

In some situations, sensors for monitoring pressure and other parameters at the dorsal surface of the foot may be provided on a sock or anklet rather than on footwear. Sensors may be mounted in locations similar to those described above with respect to footwear sensor locations, and at other locations, with conductive trace pathways communicating between sensors and trace terminations, conductive bridges, or the like. In some embodiments, the trace terminations may be located in an ankle region of the sock or anklet. FIGS. 13A, 13B illustrate a mounting band 80 and DED 100 as illustrated in FIGS. 10A-12D mounted to a sock in an ankle region. The band portion of the mounting tab may be attached to the sock near an ankle region, traversing a front portion of the ankle region. The DED is positioned in a DED receiving cavity and is positioned laterally, on one side or the other, of the front or back side of the ankle region.

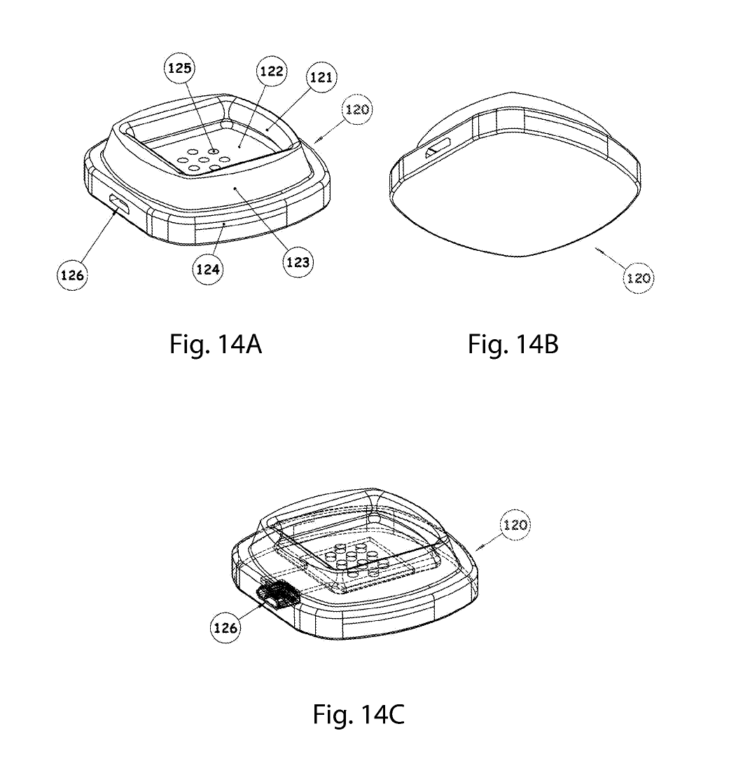

In some embodiments, DEDs as described herein may support wireless charging. In these embodiments, DEDs may be semi-permanently or permanently associated with an underlying substrate. In some embodiments, a DED charging device may be provided. FIGS. 14A-14C show one embodiment of a charging station 120 for charging a DED component 100 having the configuration illustrated in FIGS. 11A-11B. The configuration of charging station 120, as illustrated, is similar to the configuration of DED receiving portion 82 of mounting tab 80. Exposed interface surfaces of conductive pins 125 are located on an exposed surface of DED receipt cavity 121, on a lower, internal cavity surface 122 in the embodiment shown in FIG. 14A. The upper and side wall contours of DED receipt cavity 121 correspond to the outer contours of DED body, as shown in FIGS. 11A, 11B, to provide detachable yet stable mounting of DED body portion 110 within DED receipt cavity 121 of charging station 120. Exterior surfaces of DED receipt cavity 121 may be provided as tapered external side walls 123. Charger base 124 provides stable positioning of the charging station 120 and locates electrical charging interface 126.

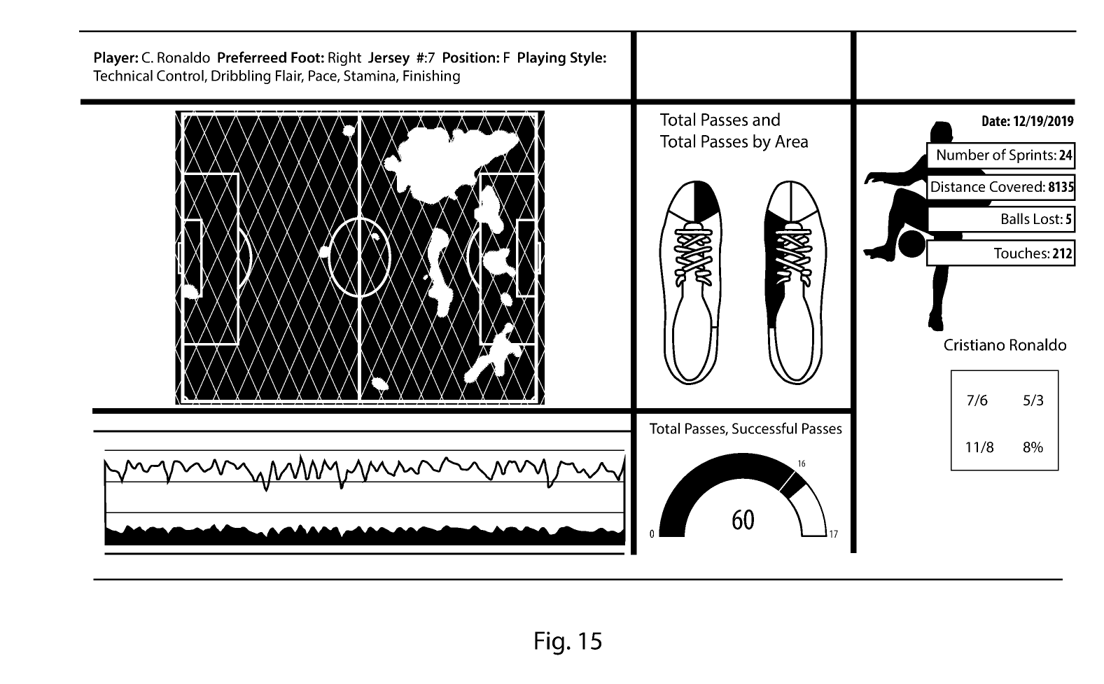

In some embodiments, footwear as described herein may be provided in the form of sports shoes, such as soccer shoes. Footwear, such as soccer shoes, having pressure sensors associated with a dorsal or upper part of the shoe, in combination with a DED having instrumentation such as one or more accelerometer(s), gyroscope(s), magnetometer(s), inertial measurement unit(s), or the like, associated with the shoe (e.g., in the heel area or plantar area), or with a sock, or with another user-worn accessory, may collect data including one or more of the following: ground speed and distance; acceleration and deceleration; number of passes and shots, as well as location on the foot where the pass or shot originated; passes by technique, e.g., pass/shot, on the ground or in the air, straight, hook or slice, ship or heel; force of impact with the ball; limb movement in space; dead reckoning of the player on the field (e.g., reconstruction of the path of the player on the field during the game). Footwear additionally having pressure sensors associated with a plantar part of the shoe, or footwear used in combination with socks having pressure sensors associated with a plantar part of the shoe, used in combination with a DED as described, may additionally provide data relating to running and/or movement type, efficiency, cadence, foot strike areas, and the like. FIG. 15 shows exemplary displays that may be provided using footwear as described herein, with the display providing one or more of the following types of information: number of sprints; distance covered; ball touches; total passes and passes by type; balls lost; shots made; dribbling distance and time; and the like. Graphical presentations of this information may be provided, as shown, with different colors indicating higher or lower frequency of the player's presence in certain areas of the field, frequency of ball touches, passes, and the like. Additionally, various compilations and indices of data may be presented, such as player intensity, stamina, defensive and offensive efficiency, technical control, time of possession, dribbling flair, finishing, and the like.

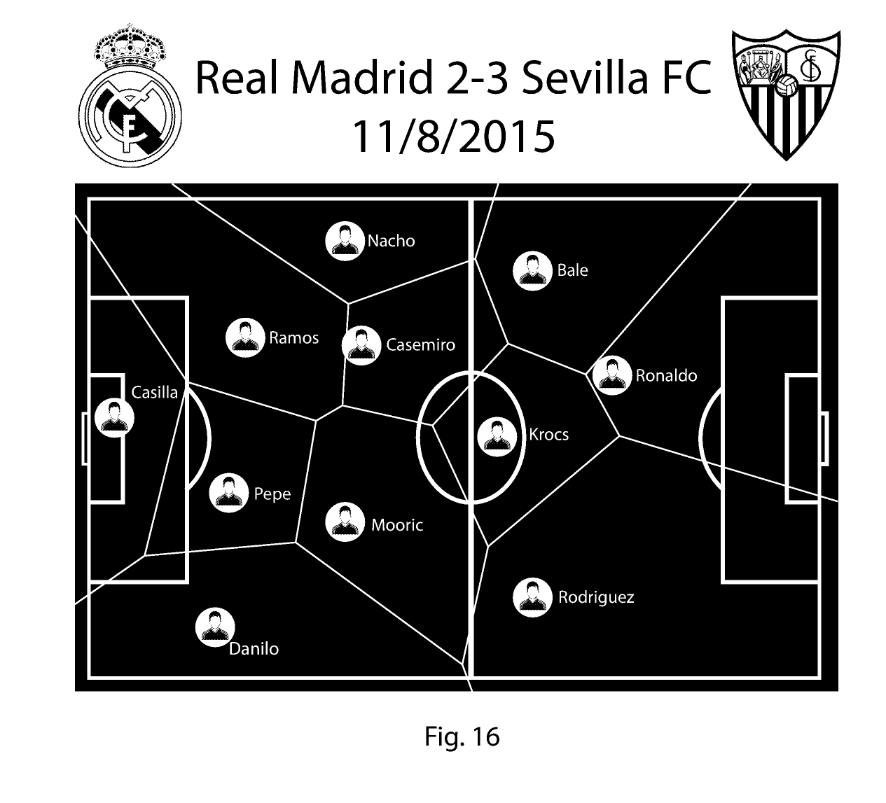

FIG. 16 illustrates a visual presentation of similar types of data for team members. These types of data, data compilations, visual presentations, and the like, may be used to produce aggregated data on a whole match (and on multiple matches) by individual players, and individual player data may be combined to provide data on teams as a whole. Combining and correlating the data across multiple players may also provide sports statistics relating to matches, replacing or supplementing manual statistics, providing more easily distributed statistics to individuals, coaches and spectators in near-real-time, and extending the affordability of stats tracking to all games, including professional and amateur games. Individual and aggregated team data may also be useful as a training and coaching tool, providing real-time analysis, post-match stats, as well as practice stats, allowing comparison of player performances and interaction, and providing useful data that contributes to making team line-ups, as well as pre-game, during-game and post-game strategies.

While sensor systems and accessories are described herein with respect to footwear, and shoes in particular, it will be appreciated that such sensor systems and accessories may be implemented in other types of footwear, including other types of sports shoes (e.g., basketball shoes, volleyball shoes, baseball shoes, tennis shoes, biking shoes and other types of sports shoes, as well as boots such as ski boots, hiking boots, and the like. Footwear and footwear-associated garments (such as socks and sporting accessories), as described, may also be used in conjunction with other sensor-enabled garments or accessories, such as heart-rate monitors, respiration monitors, heart-rate variability monitors, sensors measuring VO2max, torso acceleration, sweat volume and/or content, and the like, to provide even more comprehensive individual data.

While specific examples of sensor systems and sensor system components, such as sensors, traces, conductive terminals and mounting bands are described with reference to a footwear form factor, it will be appreciated that the features and components disclosed herein may be used with (and/or applied to) other types of wearable garments (e.g., socks, underwear, t-shirts, trousers, tights, leggings, body suits, leotards, hats, gloves, bands, and the like), and many other types of substrates. Dedicated electronic devices having different configurations may be designed to interface with a variety of sensor systems embodied in different types of footwear, garments and other types of substrates. The type of sensor(s), footwear garment(s), substrate(s), placement of sensor(s), DED, conductive terminal(s), and the like, may be varied for use in many different sensor system applications.

While the present invention has been described above with reference to the accompanying drawings in which specific embodiments are shown and explained, it is to be understood that persons skilled in the art may modify the embodiments described herein without departing from the spirit and broad scope of the invention. Accordingly, the descriptions provided above are considered as being illustrative and exemplary of specific structures, aspects and features within the broad scope of the present invention and not as limiting the scope of the invention. The various embodiments described herein may be combined to provide further embodiments. The described devices, systems and methods may omit some elements or acts, may add other elements or acts, or may combine the elements or execute the acts in a different order than that illustrated, to achieve various advantages of the disclosure. These and other changes may be made to the disclosure in light of the above detailed description.

In the present description, where used, the terms “about” and “consisting essentially of” mean ±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives, unless otherwise expressly indicated. As used herein, the terms “include” and “have” and “comprise” are used synonymously, and those terms, and variants thereof, are intended to be construed as non-limiting. In general, in the following claims, the terms used should not be construed to limit the disclosure to the specific embodiments disclosed in the specification.

1. An article of footwear including a sensor system comprising a non-electrically conductive substrate having at least one sensor associated therewith, the at least one sensor being in electrical communication with at least one electrically conductive trace that terminates at or in proximity to a signal transfer terminal, and a dedicated electronic device (DED) having signal receipt terminals that mate with signal transfer terminals and a housing component, wherein the at least one sensor is a conductive or resistive e-textile sensor located in a dorsal and/or lateral area of the footwear and the DED comprises a device selected from the group consisting of: an accelerometer; a gyroscope; an orientation sensing component; a location sensing component; an inertial measurement unit (IMU); a magnetometer; and a temperature sensor.

2. (canceled)

3. The article of footwear of claim 1, wherein the DED housing component is detachably mountable to the footwear.

4. The article of footwear of claim 1, additionally comprising at least one conductive bridge at each conductive trace termination.

5. The article of footwear of claim 4, wherein the at least one conductive bridge is fabricated from conductive thread or fabric.

6. The article of footwear of claim 4, wherein the at least one conductive bridge is fabricated from a conductive thermoplastic elastomer or a conductive ink or a conductive metallic material.

7. The article of footwear of claim 1, wherein at least one conductive contact is mounted to a non-electrically conductive tab and the non-electrically conductive tab is mounted to the footwear.

8. (canceled)

9. The article of footwear of claim 7, wherein the non-electrically conductive tab comprises a band portion having the at least one exposed contact and a DED receipt cavity sized and configured to receive the DED housing component.

10. (canceled)

11. (canceled)

12. (canceled)

13. (canceled)

14. The article of footwear of claim 1, wherein the article of footwear is a sports shoe or a sports sock or a sports footwear accessory.

15. (canceled)

16. The article of footwear of claim 1, wherein the article of footwear is a boot.

17. The article of footwear of claim 1, wherein the at least one sensor provides data relating to force exerted against specific sensor locations.

18. (canceled)

19. The article of footwear of claim 1, wherein the non-electrically conductive substrate is a shoe or a boot or a shin guard.

20. An article of footwear having a sensor system comprising: a non-electrically conductive substrate having a plurality of sensors associated therewith at a plurality of dorsal and/or lateral locations on the article of footwear, each of the sensors being in electrical communication with at least one electrically conductive trace that terminates at or in proximity to a trace termination located on the article of footwear or on an associated garment or accessory.

21. The article of footwear of claim 20, comprising at least one sensor selected from the following: a conductive electrode; a conductive sensor; a resistive sensor; a pressure or force sensor; a temperature sensor; a galvanic skin response sensor; a moisture sensor; a heart rate sensor; a respiration sensor; an electromyography sensor; a blood gas content sensor; a skin conductivity sensor; an accelerometer; a gyroscope; and a location or position sensor.

22. The article of footwear of claim 20, having at least one sensor comprising a resistive e-textile sensor.

23. The article of footwear of claim 20, having at least one sensor fabricated from a conductive or resistive thermoplastic elastomer or a conductive or resistive ink or a conductive or resistive metallic material.

24. The article of footwear of claim 20, additionally comprising a DED in electrical contact with the at least one sensor, wherein the DED comprises a device selected from the group consisting of: an accelerometer; a gyroscope; an orientation sensing component; a location sensing component; an inertial measurement unit (IMU); a magnetometer; and a temperature sensor.

25. (canceled)

26. (canceled)

27. (canceled)

28. The article of footwear of claim 20, wherein the substantially non-electrically conductive substrate is a sock.

29. The article of footwear of claim 20, comprising a sports shoe or a boot.

30. (canceled)

31. A system comprising at least one sensor associated with an article of footwear selected from the following: a conductive electrode; a conductive sensor; a resistive sensor; a pressure or force sensor; a temperature sensor; a galvanic skin response sensor; a moisture sensor; a heart rate sensor; a respiration sensor; an electromyography sensor; a blood gas content sensor; a skin conductivity sensor; an accelerometer; a gyroscope; and a location or position sensor and at least one DED in electrical contact with the at least one sensor, wherein the DED comprises a device selected from the group consisting of: an accelerometer; a gyroscope; an orientation sensing component; a location sensing component; an inertial measurement unit (IMU); a magnetometer; and a temperature sensor; wherein the system collects and is capable of reporting data including one or more of the following user parameters during or following a user's use of the system: ground speed and distance; acceleration and deceleration; number of passes and shots, as well as location on the foot where the pass or shot originated; passes by technique, e.g., pass/shot, on the ground or in the air, straight, hook or slice, ship or heel; force of impact with the ball; limb movement in space; dead reckoning of the user on the field.

32. (canceled)

33. (canceled)

34. (canceled)

35. The system of claim 31, wherein the at least one pressure sensor is fabricated from at least one of the following: a conductive or resistive e-textile material; a conductive or resistive thermoplastic elastomer; a conductive or resistive ink material; and a conductive or resistive metallic material.
